(19) United States
(12) Patent Application Publication
(10) Pub. No.: US 2024/0384257 A1
(43) Pub. Date: Nov. 21, 2024

(54) NUCLEIC ACID EXTRACTION COMPOSITION, NUCLEIC ACID EXTRACTION DEVICE AND NUCLEIC ACID EXTRACTION METHOD

(71) Applicants: Chengdu BOE Optoelectronics Technology Co., Ltd., Chengdu, Sichuan (CN); BOE Technology Group Co., Ltd., Beijing (CN)

(72) Inventor: Zhihong WU, Beijing (CN)

(21) Appl. No.: 18/578,679

(22) PCT Filed: Sep. 29, 2021
(86) PCT No.: PCT/CN2021/121722

§ 371 Date: Jan. 11, 2024

(51) Int. Cl. C12N 15/10

(52) U.S. Cl. CPC C12N 15/1013

(57) ABSTRACT

The invention provides a nucleic acid extraction composition, a nucleic acid extraction device and a nucleic acid extraction method. The nucleic acid extraction composition comprises: a magnetic bead liquid, wherein the magnetic bead liquid comprises magnetic beads and a first group connected with the magnetic beads; the buffer solution system comprises digestive juice, protease liquid and binding liquid; wherein the digestive juice and the binding liquid are separated from each other, and the digestive juice comprises chaotropic salt; the binding liquid comprises a decomposer for exposing an organic group in the nucleic acid, so that the organic group is bound with the first group in the nucleic acid extraction process.

### CROSS REFERENCE TO RELATED APPLICATIONS

This application is a national phase entry under 35 U.S.C. § 371 of International Application No. PCT/CN2021/121722, filed on Sep. 29, 2021, the entire content of which is incorporated herein by reference.

### TECHNICAL FIELD

The disclosure relates to the technical field of nucleic acid extraction, in particular to a nucleic acid extraction composition, a nucleic acid extraction device and a nucleic acid extraction method.

### BACKGROUND

Circulating cell-free DNA (cfDNA) refers to a DNA fragment that is present in human blood circulation and is free outside cells, with short fragments and low content. At present, a specific mechanism of cfDNA production is still unclear in the scientific community. A relatively accepted view is that cfDNA is primarily derived from DNA fragments released by apoptosis. In recent years, research has found that the size distribution of cfDNA fragments in blood of cancer patients differs from that of normal human samples. High sensitivity detection of many different types of tumors can be performed by simple low-depth sequencing, which offers a new idea for early cancer screening at lower cost. Currently, commercially available cfDNA detection products developed for prenatal disgnosis (Non-invasive Prenatal Testing, NIPT) have been put on the market, indicating that the study of a cfDNA fragmentation law has a high potential application value.

### SUMMARY

The disclosure provides a nucleic acid extraction composition, a nucleic acid extraction device and a nucleic acid extraction method. The nucleic acid extraction composition includes: a magnetic bead solution, including magnetic beads and a first group linked to the magnetic beads; and a buffer system, including a digestion solution, a protease solution and a binding solution. The digestion solution is separated from the binding solution, and includes a chaotropic salt. The binding solution includes a resolvent for exposing an organic group in a nucleic acid to allow the organic group to bind to the first group during nucleic acid extraction.

In a possible embodiment, the resolvent includes isopropanol, and the first group includes silicon hydroxyl.

In a possible embodiment, the digestion solution further includes: a chelating agent, a surfactant, and a buffer salt.

In a possible embodiment, in the digestion solution, the concentration of the chaotropic salt is in the range of 1 M to 6 M, the concentration of the chelating agent is in the range of 10 mM to 100 mM, the volume percentage of the surfactant is in the range of 10% to 30%, the concentration of the buffer salt is in the range of 30 mM to 100 mM and a pH of the buffer salt is in the range of 7.5 to 8.5.

In a possible embodiment, the binding solution further includes: a chaotropic salt, a chelating agent, a surfactant, and a buffer salt.

In a possible embodiment, in the binding solution, the concentration of the chaotropic salt is in the range of 2 M to 5 M, the volume percentage of the resolvent is in the range of 20% to 50%, and the concentration of the chelating agent is in the range of 1 mM to 5 mM; the volume percentage of the surfactant is in the range of 1% to 5%; and the concentration of the buffer salt is in the range of 10 mM to 100 mM, and a pH of the buffer salt is in the range of 7.5 to 8.5.

In a possible embodiment, the protease solution includes: proteinase K, a buffer salt, a soluble salt, and glycerol.

In a possible embodiment, in the protease solution, the concentration of the proteinase K is in the range of 10 mg/ml to 30 mg/ml; the concentration of the buffer salt is in the range of 30 mM to 100 mM and a pH of the buffer salt is in the range of 7.5 to 8.5, and the concentration of the soluble salt is in the range of 1 mM to 5 mM; and the volume percentage of the glycerol is in the range of 15% to 55%.

In a possible embodiment, the buffer system further includes a first washing solution and a second washing solution. The first washing solution includes: a chaotropic salt, the resolvent, a surfactant, a buffer salt, and a chelating agent. The second washing solution includes a buffer salt, a chelating agent, and an alcohol.

In a possible embodiment, in the first washing solution, the concentration of the chaotropic salt is in the range of 1 M to 5 M, the volume percentage of the resolvent is in the range of 10% to 50%, and the concentration of the chelating agent is in the range of 1 mM to 8 mM; the volume percentage of the surfactant is in the range of 1% to 8%; and the concentration of the buffer salt is in the range of 10 mM to 100 mM, and a pH of the buffer salt is in the range of 5.5 to 6.5.

In a possible embodiment, in the second washing solution, the concentration of the buffer salt is in the range of 45 mM to 55 mM, and a pH of the buffer salt is in the range of 7.5 to 8.5, the concentration of the chelating agent is in the range of 0.4 mM to 0.6 mM, and the volume percentage of the alcohol is in the range of 75% to 85%.

In a possible embodiment, the buffer system further includes an eluent. The eluent includes enzyme-free water or a TE buffer.

In a possible embodiment, the chaotropic salt includes guanidinium isothiocyanate, guanidinium thiocyanate, guanidinium hydrochloride, urea, sodium iodide, potassium iodide, potassium chloride, sodium perchlorate or trichloroacetate; the chelating agent includes: ethylene diamine tetraacetic acid, citrate, ethylene glycol bis (2-aminoethyl ether) tetraacetic acid, and cyclohexanediaminetetraacetic acid; the surfactant includes Tween 20, Tween 21, Tween 40, Tween 60, Tween 80, Tween 85, polyethylene glycol octylphenyl ether, ethylphenylpolyethylene glycol, polyoxyethylene lauryl ether, or sorbitan monolaurate; and the buffer salt includes: tris (hydroxymethyl) aminomethane or methylmethane.

In a possible embodiment, the chaotropic salt includes guanidinium isothiocyanate; the chelating agent includes ethylene diamine tetraacetic acid; the surfactant includes: Tween 20; and the buffer salt includes: tris (hydroxymethyl) aminomethane or methylmethane.

In a possible embodiment, the magnetic beads have a size ranging from 200 nm to 5 μm.

Embodiments of the disclosure further provide a nucleic acid extraction device, including the nucleic acid extraction composition provided in the embodiments of the disclosure.

Embodiments of the disclosure further provide a nucleic acid extraction method, including: obtaining a plasma sample according to a blood sample to be tested; adding a protease solution and a digestion solution into the plasma sample, carrying out a reaction under heating for a first period of time, and performing cooling to separate a nucleic acid from the plasma sample; adding a binding solution to the plasma sample from which the nucleic acid is separated to expose an organic group in the nucleic acid by a resolvent in the binding solution; adding a magnetic bead solution into the plasma sample exposing the organic group to link the nucleic acid to the magnetic beads by binding a first group linked to magnetic beads in the magnetic bead solution to the organic group, so as to separate the magnetic beads linked to the nucleic acid from the plasma sample; and separating the magnetic beads from the nucleic acid.

In a possible embodiment, the adding the protease solution and the digestion solution into the plasma sample, carrying out the reaction under heating for a first period of time, and performing cooling includes: adding the protease solution into the plasma sample, and performing uniform mixing; adding the digestion solution into the plasma sample in which the protease solution is added, performing uniform mixing, and performing incubation at a temperature of 40° C.-60° C. for 20 min-40 min; and placing the plasma sample after the incubation on ice to be cooled for 1 min-10 min, or wait to be cooled to room temperature.

In a possible embodiment, the adding the magnetic bead solution into the plasma sample exposing the organic group to link the nucleic acid to the magnetic beads by binding the first group linked to the magnetic beads in the magnetic bead solution to the organic group, so as to separate the magnetic beads linked to the nucleic acid from the plasma sample includes: adding the magnetic bead solution at a volume percentage ratio of the magnetic bead solution to the plasma sample, which is a first ratio, and performing uniform mixing; performing centrifugation, placing the plasma sample mixed with the magnetic bead solution on a magnetic frame, and standing for 1 min-10 min; removing a supernatant; adding a first washing solution, standing, removing the supernatant after a supernatant is clarified, and repeating the above operations for multiple times; and adding a second washing solution, performing uniform mixing, performing centrifugation, placing the centrifuged solution on a magnetic frame for 1 min-10 min, removing the supernatant after a supernatant is clarified, and repeating the above operations for multiple times.

In a possible embodiment, the separating the magnetic beads from the nucleic acid includes: adding an eluent, performing uniform mixing, and standing for 5 min-20 min; and performing centrifugation, and placing the centrifuged solution on a magnetic frame for 1 min-10 min to obtain a supernatant.

In a possible embodiment, the obtaining the plasma sample according to the blood sample to be tested includes: centrifuging the blood sample at a temperature of 1° C.-10° C. for 5 min-20 min; obtaining a supernatant after the centrifuging to form an initial plasma sample; centrifuging the initial plasma sample at a temperature of 1° C.-10° C. for 5 min-50 min; and obtaining a supernatant to form the plasma sample.

### BRIEF DESCRIPTION OF FIGURES

FIG. 1 is a first schematic diagram of a nucleic acid extraction composition according to embodiments of the disclosure.

FIG. 2 is a second schematic diagram of the nucleic acid extraction composition according to embodiments of the disclosure.

FIG. 3 is a third schematic diagram of the nucleic acid extraction composition according to embodiments of the disclosure.

FIG. 4 shows a comparison between nucleic acid extraction purity provided by embodiments of the disclosure and extraction purity obtained by a commercially available nucleic acid extraction composition.

FIG. 5 shows a comparison of extraction amounts for different nucleic acid fragments via the nucleic acid extraction composition according to embodiments of the disclosure.

FIG. 6 shows a comparison of nucleic acid extraction for different volumes of blood samples by the nucleic acid extraction composition according to embodiments of the disclosure.

FIG. 7 is a schematic diagram of distribution of nucleic acids of different length fragments obtained by extracting a normal human plasma sample by using a commercially available nucleic acid extraction kit.

FIG. 8 illustrates extracting a normal human plasma sample by using a kit containing the nucleic acid extraction composition according to embodiment of the disclosure.

FIG. 9 is a first schematic flow diagram of a nucleic acid extraction method according to embodiments of the disclosure.

FIG. 10 shows a comparison between results obtained by using nucleic acid extraction compositions of different proportions according to embodiments of the disclosure.

FIG. 11 shows another comparison between results obtained by using nucleic acid extraction compositions of different proportions according to embodiments of the disclosure.

### DETAILED DESCRIPTION

In order to make the objectives, technical solutions, and advantages of the embodiments of the disclosure clearer, the technical solutions of the embodiments of the disclosure will be described clearly and completely below with reference to the accompanying drawings of the embodiments of the disclosure. Apparently, the described embodiments are some, not all, of the embodiments of the disclosure. Based on the described embodiments of the disclosure, all other embodiments obtained by those of ordinary skill in the art without inventive efforts fall within the scope of protection of the disclosure.

Unless otherwise indicated, the technical or scientific terms used in the disclosure should have a general meaning understood by those of ordinary skill in the art to which the disclosure belongs. The terms “first,” “second,” and the like used in the disclosure do not denote any order, quantity, or importance, but are merely used to distinguish different components. “Include” or “comprise” and other similar words mean that an element or item preceding the word covers elements or items and their equivalents listed behind the word without excluding other elements or items. “Connection” or “connected” and the like are not limited to physical or mechanical connection, but can include electrical connection, whether direct or indirect. “Upper”, “lower”, “left”, “right” and the like are only used for representing a relative position relation, and when an absolute position of the described object is changed, the relative position relation can also be correspondingly changed.

In order to keep the following description of the embodiments of the disclosure clear and concise, the disclosure omits detailed descriptions of known functions and known components.

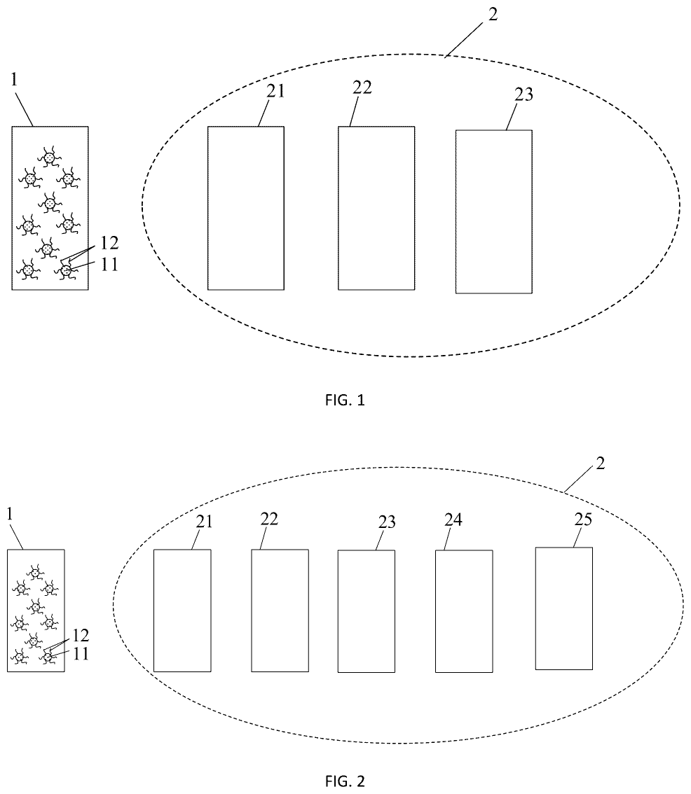

Referring to FIG. 1, embodiments of the disclosure provide a nucleic acid extraction composition, including:
a magnetic bead solution 1, including magnetic beads 11, and a first group 12 linked to the magnetic beads 11; and
a buffer system 2, including: a digestion solution 21, a protease solution 23, and a binding solution 22 which are separated; where the digestion solution 21 is separated from the binding solution 22, and in particular, the protease solution 23, the digestion solution 21, and the binding solution 22 may be separated from each other, and the digestion solution 21 includes a chaotropic salt; and the binding solution 22 includes a resolvent for exposing an organic group in a nucleic acid to allow the organic group to bind to the first group 12 during nucleic acid extraction.

In the embodiments of the disclosure, the buffer system 2 includes the digestion solution 21 and the binding solution 22 which are separated from each other, during extraction of a nucleic acid in a blood sample by using the nucleic acid extraction composition, cells in a plasma sample may first be lysed via the chaotropic salt in the digestion solution 21 for subsequent extraction of a nucleic acid in the cells, the nucleic acid is separated by the protease solution 23, then the binding solution 22 is added, the organic group in the nucleic acid can be exposed by the resolvent in the binding solution 22, the magnetic bead solution 1 is then added, the first group 12 linked to the magnetic beads 11 in the magnetic bead solution 1 can bind to the organic group, thereby linking the nucleic acid to the magnetic beads 11, based on the adsorption of the magnetic beads 11, other substances may be removed from the blood sample by washing and finally a relatively pure nucleic acid is obtained, thus realizing the separation of the nucleic acid from the blood sample. In the embodiments of the disclosure, the digestion solution 21 is separated from the binding solution 22, and the digestion solution 21 and the binding solution 22 are added separately for a reaction, so that a heating process during which the digestion solution 21 is added is separated from a process of binding the magnetic beads 11 to the nucleic acid by adding the binding solution 22, and the problem that the magnetic beads 11 attract each other during the heating process, impurities are not easily removed by washing in a later stage, and then the obtained nucleic acid has a low purity is avoided. Moreover, the binding solution 22 contains the resolvent, and the resolvent can expose the organic group in the nucleic acid, so that the magnetic beads 11 are linked to the nucleic acid by binding the organic group to the first group, and this binding allows to extract large nucleic acid fragments and to extract small nucleic acid fragments, ameliorating the disadvantage that only large nucleic acid fragments can be extracted in the related art. In addition, the digestion solution and the binding solution have different components and are separated, facilitating adjustment of a ratio of addition of the digestion solution and the binding solution according to blood samples of different volumes, improving the problem of poor operability on the addition volume of extraction solution for nucleic acid extraction under different blood sample volumes.

In a possible embodiment, the resolvent includes isopropanol, and the first group includes silicon hydroxyl. In the embodiments of the disclosure, isopropanol may expose the organic group (e.g., a basic group may be included) in the nucleic acid. The first group includes silicon hydroxyl, and the magnetic beads linked to silicon hydroxyl bind to the nucleic acid by hydrogen bonding and a van der Waals force (specifically, silicon hydroxyl may bind to the basic group on a cfDNA strand through hydrogen bonding; and silicon hydroxyl may bind to other organic groups on the cfDNA strand through the van der Waals force), so that the bias in the selection of nucleic acid fragment lengths may be reduced, and the problem that when polyethylene glycol (PEG) is used to aggregate and precipitate the nucleic acid based on dehydration and a spatial exclusion principle, extraction of small fragments may not be facilitated during extraction of different DNA fragments for low-concentration nucleic acid solutions such as circulating free DNA (cfDNA), thereby creating a certain bias is avoided. For the research work of cfDNA, detection of small fragment DNA (for example, cfDNA of about 100 bp and 170 bp) is more valuable in clinical application. Moreover, when polyvinene glycol (PEG) is used to separate out the nucleic acid by aggregating and precipitate the nucleic acid through dehydration and the spatial exclusion principle, the operability on the addition volume of extraction solution for nucleic acid extraction under different blood sample volumes is often poor.

In a possible embodiment, the digestion solution 21 further includes: a chelating agent, a surfactant, and a buffer salt. In particular, the chelating agent may be used to stabilize the nucleic acid, the surfactant may be used to disperse the magnetic beads to prevent the magnetic beads from binding to each other, and the buffer salt may be used to regulate a pH of the digestion solution.

Specifically, in the digestion solution 21, the concentration of the chaotropic salt is in the range of 2M to 5M, the volume percentage of the resolvent is in the range of 20% to 50%, and the concentration of the chelating agent is in the range of 1 mM to 5 mM; the volume percentage of the surfactant is in the range of 1% to 5%; and the concentration of the buffer salt is in the range of 10 mM to 100 mM, and a pH of the buffer salt is in the range of 7.5 to 8.5. In particular, the buffer salt may have a pH of 8.0, and the digestion solution in this ratio may allow the nucleic acid extraction composition to have a high nucleic acid extraction rate.

In particular, in the digestion solution 21, the chaotropic salt includes guanidinium isothiocyanate (GITC), guanidinium thiocyanate, guanidinium hydrochloride, urea, sodium iodide, potassium iodide, potassium chloride, sodium perchlorate or trichloroacetate; the chelating agent includes: ethylene diamine tetraacetic acid, citrate, ethylene glycol bis (2-aminoethyl ether) tetraacetic acid, and cyclohexanediaminetetraacetic acid; the surfactant includes: Tween 20, Tween 21, Tween 40, Tween 60, Tween 80, Tween 85, polyethylene glycol octylphenyl ether (Triton x-100), ethylphenylpolyethylene glycol (NP-40), polyoxyethylene lauryl ether (Brij30), or sorbitan monolaurate (Span20); and the buffer salt includes: tris (hydroxymethyl) aminomethane (Tris) or methylmethane (Bis-Tris).

Specifically, in the digestion solution 21, the chaotropic salt includes guanidinium isothiocyanate (GITC), the chelating agent may include ethylene diamine tetraacetic acid (EDTA), the buffer salt may include tris (hydroxymethyl) aminomethane (THAM, Tris), and the surfactant may include Tween 20. In the embodiments of the disclosure, ethylene diamine tetraacetic acid can stabilize the nucleic acid, tris (hydroxymethyl) aminomethane can regulate a pH in the digestion solution 21, and Tween 20, as a surfactant, can better disperse the magnetic beads into a solution to bind to DNA, so as to better capture the nucleic acid.

In a possible embodiment, the binding solution 22 further includes: a chaotropic salt, a chelating agent, a surfactant, and a buffer salt. The chelating agent can be used to stabilize the nucleic acid, the surfactant can be used to disperse the magnetic beads, and the buffer salt can be used to regulate a pH of the binding solution.

In a possible embodiment, in the binding solution 22, the concentration of the chaotropic salt is in the range of 2 M to 5 M, the volume percentage of the resolvent is in the range of 20% to 50%, and the concentration of the chelating agent is in the range of 1 mM to 5 mM; the volume percentage of the surfactant is in the range of 1% to 5%; and the concentration of the buffer salt is in the range of 10 mM to 100 mM, and a pH of the buffer salt is in the range of 7.5 to 8.5. In particular, the buffer salt may have a pH of 8.0, and the binding solution 22 in this ratio may allow the nucleic acid extraction composition to have a high nucleic acid extraction rate.

In particular, in the binding solution 22, the chaotropic salt includes guanidinium isothiocyanate (GITC), guanidinium thiocyanate, guanidinium hydrochloride, urea, sodium iodide, potassium iodide, potassium chloride, sodium perchlorate or trichloroacetate; the chelating agent includes: ethylene diamine tetraacetic acid, citrate, ethylene glycol bis (2-aminoethyl ether) tetraacetic acid, and cyclohexanediaminetetraacetic acid; the surfactant includes: Tween 20, Tween 21, Tween 40, Tween 60, Tween 80, Tween 85, polyethylene glycol octylphenyl ether (Triton x-100), ethylphenylpolyethylene glycol (NP-40), polyoxyethylene lauryl ether (Brij30), or sorbitan monolaurate (Span20); and the buffer salt includes: tris (hydroxymethyl) aminomethane (Tris) or methylmethane (Bis-Tris).

In particular, in the binding solution 22, the chaotropic salt includes guanidinium isothiocyanate (GITC), the chelating agent includes ethylene diamine tetraacetic acid, the buffer salt includes tris (hydroxymethyl) aminomethane, and the surfactant includes Tween 20. In the embodiments of the disclosure, ethylene diamine tetraacetic acid can stabilize the nucleic acid, tris (hydroxymethyl) aminomethane can regulate a pH in the binding solution 22, and Tween 20, as a surfactant, can better disperse the magnetic beads into a solution for binding to and capturing DNA.

In a possible embodiment, the protease solution includes: proteinase K, a buffer salt, a soluble salt, and glycerol. Specifically, a proteinase K can digest protein molecules in plasma, thereby releasing cfDNA bound to the protein molecules; the buffer salt can be used to regulate a pH of the protease solution; the soluble salt can facilitate protease digestion; and the glycerol can prevent protease from becoming ineffective after long-term storage.

In a possible embodiment, in the protease solution, the concentration of the proteinase K is in the range of 10 mg/mL to 30 mg/ml; the concentration of the buffer salt is in the range of 30 mM to 100 mM and a pH of the buffer salt is in the range of 7.5 to 8.5, and the concentration of the soluble salt is in the range of 1 mM to 5 mM; and the volume percentage of the glycerol is in the range of 15% to 55%, and the protease solution in this ratio may allow the nucleic acid extraction composition to have a high nucleic acid extraction rate.

In particular, in the protease solution, the buffer salt may include tris (hydroxymethyl) aminomethane (THAM, Tris) and the soluble salt may include CaCl₂).

In a possible embodiment, with reference to FIG. 2, the buffer system further includes a washing solution 24, and the washing solution 24 in particular may include: a first washing solution 241 and a second washing solution 242; the first washing solution 241 includes: a chaotropic salt, s resolvent, a surfactant, a buffer salt, and a chelating agent; and the second washing solution 242 includes a buffer salt, a chelating agent, and an alcohol. The first washing solution 241 has the same composition as the binding solution 22, but the concentrations of guanidinium isothiocyanate (GITC), and the resolvent (isopropanol) are relatively small, and the concentrations of the buffer salt (Tween 20), the buffer salt (Bis-Tris pH 6.0), and the chelating agent (EDTA) are relatively high, so that the first washing solution 241 can remove unwanted proteins by washing while maintaining stability of the nucleic acid; and the second washing solution 242 may remove at least a portion of substances in the first washing solution 241 by washing (e.g., the resolvent in the first washing solution 241 is removed by washing) while maintaining the stability of the nucleic acid to prevent the nucleic acid from being hydrolyzed by DNase.

Specifically, in the first washing solution 241, the concentration of the chaotropic salt is in the range of 1 M to 5 M, the volume percentage of the resolvent is in the range of 10% to 50%, and the concentration of the chelating agent is in the range of 1 mM to 8 mM; the volume percentage of the surfactant is in the range of 1% to 8%; and the concentration of the buffer salt is in the range of 10 mM to 100 mM and a pH of the buffer salt is in the range of 5.5 to 6.5, and in particular, the buffer salt may have a pH of 6.0; and in the second washing solution 242, the concentration of the buffer salt is in the range of 45 mM to 55 mM and a pH of the buffer salt is in the range of 7.5 to 8.5, the concentration of the chelating agent is in the range of 0.4 mM to 0.6 mM, and the volume percentage of the alcohol is in the range of 75% to 85%; and the first washing solution 241 and the second washing solution 242 in this ratio may allow the nucleic acid extraction composition to have a high nucleic acid extraction rate.

In particular, in the first washing solution 241, the chaotropic salt includes guanidinium isothiocyanate (GITC), the resolvent includes isopropanol, the chelating agent may include ethylene diamine tetraacetic acid (EDTA), the buffer salt may include methylmethane (Bis-Tris), and the surfactant includes Tween 20. In the second washing solution 242, the buffer salt includes tris (hydroxymethyl) aminomethane, the chelating agent includes ethylene diamine tetraacetic acid, and the alcohol includes ethanol (EtOH).

In a possible embodiment, with reference to FIG. 2, the buffer system further includes an eluent 25. In the embodiments of the disclosure, separation of the magnetic beads from the nucleic acid can be achieved by the eluent. In particular, the eluent may include enzyme-free water or a TE buffer.

In a possible embodiment, the magnetic beads have a size ranging from 200 nm to 5 μm, and the magnetic beads in this size range can better capture the nucleic acid.

Specifically, in the embodiments of the disclosure, components of the nucleic acid extraction composition may be specifically as follows:
the magnetic beads with silicon hydroxyl modified on the surface thereof have a size of 200 nm to 5 μm;
in the buffer system:
the digestion solution 21: 1 M-6 M GITC, 10%-30% Tween 20, 30 mM-100 mM Tris (pH 8.0), and 10 mM-100 mM EDTA;
proteinase K solution: 10 mg/mL-30 mg/ml proteinase K, 30 mM-100 mM Tris (pH 8.0), 1 mM-5 mM CaCl₂), and 15%-55% by volume glycerol;
the binding solution 22: 2 M-5 M GIT, 20%-50% isopropanol, 1%-5% Tween 20, 10 mM-100 mM Tris (pH 8.0), and 1 mM-5 mM EDTA;
the first washing solution: 1 M-5 M GITC, 10%-50% isopropanol, 1%-8% Tween 20, 10 mM-100 mM Bis-Tris (pH 6.0), and 1 mM-8 mM EDTA;
the second washing solution: 50 mM Tris (pH 8.0), 0.5 mM EDTA, and 80% by volume EtOH; and
the eluent includes enzyme-free water or TE buffer.

With reference to FIG. 4, a comparison table of the purity of nucleic acid extracted by a currently commercially available nucleic acid extraction kit and the purity of nucleic acid extracted by a kit containing a nucleic acid extraction composition provided in the embodiment of the disclosure is shown, where the corresponding three values in the commercially available nucleic acid extraction kit are the purity test results of three parallel extractions of the same sample, respectively, the three values corresponding to the kit containing the nucleic acid extraction composition provided by the embodiments of the disclosure are the purity test results of three parallel extractions of the same sample, respectively. It can be seen that the three results of the extraction by the kit containing the nucleic acid extraction composition provided by the embodiments of the disclosure have a small deviation, that is, the extraction performance of the nucleic acid extraction composition provided by the embodiments of the disclosure is stable. Compared with the three values obtained via the commercially available nucleic acid extraction kit, the purity is closer or higher, i.e., the reliability of the nucleic acid extraction result is better, and the extraction effect is better.

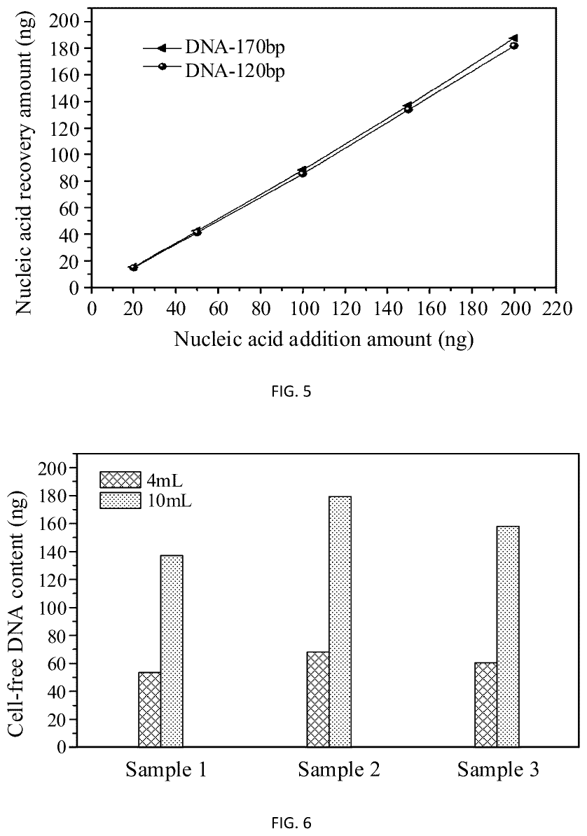

Referring to FIG. 5, different amounts of double stranded DNA of 120 bp and 170 bp are added into pure plasma. A comparative schematic diagram of recovery rates during extraction by the nucleic acid extraction composition provided by the embodiments of the disclosure is shown, and as can be seen from FIG. 5, the nucleic acid extraction composition provided by the embodiments of the disclosure has a low bias for extraction of nucleic acid fragments of different lengths, i.e., both long and short nucleic acid fragments can be extracted. Referring to FIG. 6, a comparative diagram of the amount of cell-free DNA extracted

from 4 mL of a plasma sample and 10 ml of a plasma sample by the nucleic acid extraction composition provided by the embodiments of the disclosure, as can be seen from FIG. 6, a ratio of the amount of cell-free DNA extracted from 4 mL of the plasma sample by the nucleic acid extraction composition according to the embodiments of the disclosure to the amount of cell-free DNA extracted from 10 mL of the plasma sample by the nucleic acid extraction composition according to the embodiments of the disclosure is consistent with a ratio of 4 mL of the plasma sample to 10 mL of the plasma sample, the extraction amount has the same ratio as the total volume input, small molecule fragments are not lost, and the extraction effect is relatively good for DNA extraction under different blood sample volumes.

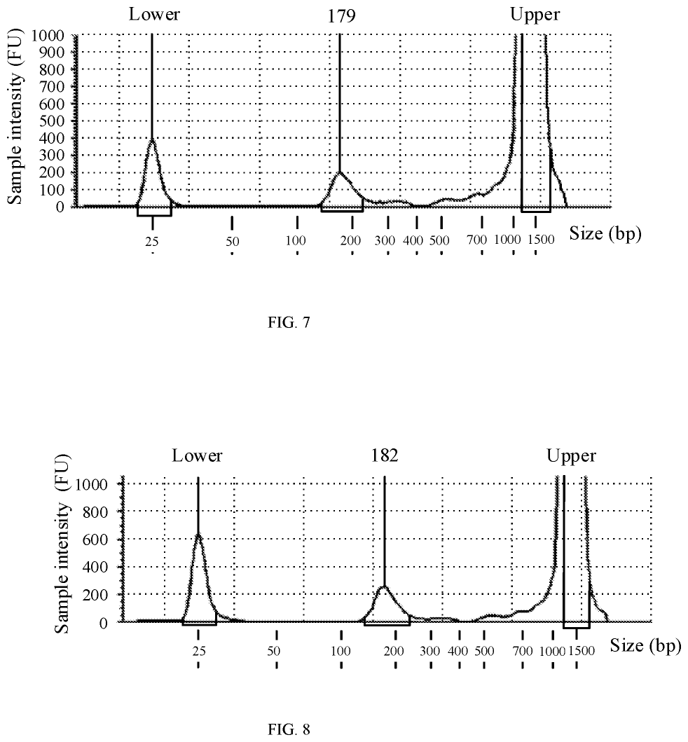

Referring to FIGS. 7 and 8, FIG. 7 is a schematic diagram of distribution of nucleic acids of different length fragments obtained by extracting a normal human plasma sample by using a commercially available nucleic acid extraction kit, FIG. 8 is a schematic diagram of distribution of nucleic acids of different length fragments obtained by extracting a normal human plasma sample by using a kit containing the nucleic acid extraction composition according to the embodiment of the disclosure, as can be seen from FIG. 7 and FIG. 8, a large-size cell-free nucleic acid extraction kit according to an embodiment of the disclosure has a higher recovery rate of cfDNA fragments with a main peak of about 167 bp than that of the currently commercially available product.

Based on the same inventive concept, an embodiment of the disclosure further provides a nucleic acid extraction device, including the nucleic acid extraction composition provided by the embodiment of the disclosure.

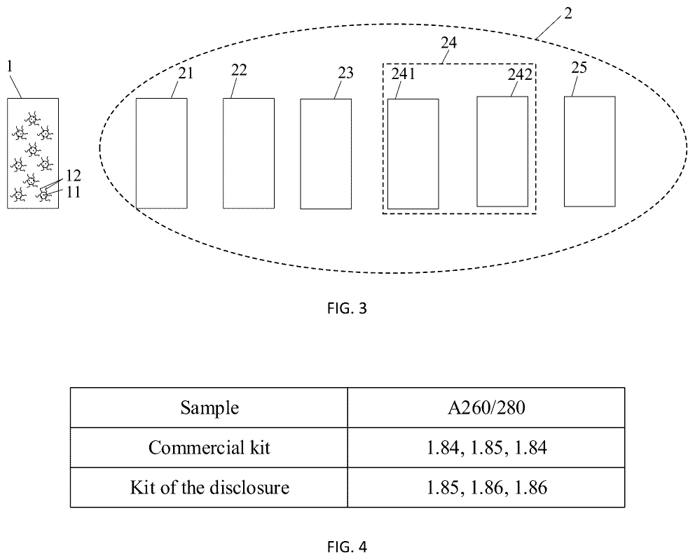

Specifically, in the embodiments of the disclosure, with reference to FIG. 3, the magnetic bead solution 1, the digestion solution 21, the binding solution 22, the proteinase K solution 23, the first washing solution 241, the second washing solution 242, and the eluent 25 may be separated from each other and added separately during nucleic acid extraction.

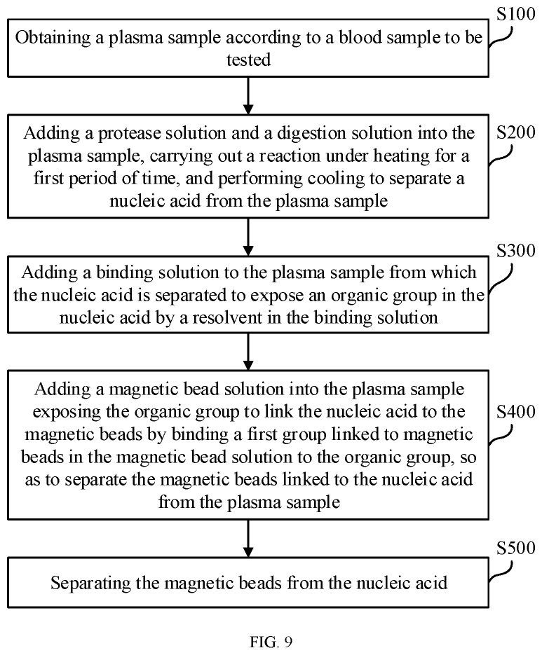

Based on the same inventive concept, referring to FIG. 9, an embodiment of the disclosure further provides a nucleic acid extraction method, including the following steps.

Step S100: obtaining a plasma sample according to a blood sample to be tested.

Specifically, obtaining the plasma sample according to the blood sample to be tested may include:
centrifuging the blood sample at a temperature of 1° C.-10° C. for 5 min-20 min;
obtaining a supernatant after the centrifuging to form an initial plasma sample;
centrifuging the initial plasma sample at a temperature of 1° C.-10° C. for 5 min-50 min; and
obtaining a supernatant to form the plasma sample.

Step S200: adding a protease solution and a digestion solution into the plasma sample, carrying out a reaction under heating for a first period of time, and performing cooling to separate a nucleic acid from the plasma sample.

In particular, adding the protease solution and the digestion solution into the plasma sample, carrying out the reaction under heating for a first period of time, and performing cooling may include:
adding the protease solution into the plasma sample, and performing uniform mixing;
adding the digestion solution into the plasma sample added with the protease solution, performing uniform mixing, and performing incubation at a temperature of 40° C.-60° C. for 20 min-40 min; and
placing the plasma sample after the incubation on ice to be cooled for 1 min-10 min, or wait to be cooled to room temperature.

Step S300: adding a binding solution to the plasma sample from which the nucleic acid is separated to expose an organic group in the nucleic acid by a resolvent in the binding solution. Step S400: adding a magnetic bead solution into the plasma sample exposing the organic group to link the nucleic acid to the magnetic beads by binding a first group linked to magnetic beads in the magnetic bead solution to the organic group, so as to separate the magnetic beads linked to the nucleic acid from the plasma sample.

Specifically, adding the magnetic bead solution into the plasma sample exposing the organic group to link the nucleic acid to the magnetic beads by binding the first group linked to the magnetic beads in the magnetic bead solution to the organic group, so as to separate the magnetic beads linked to the nucleic acid from the plasma sample may include:
adding the magnetic bead solution at a volume percentage ratio of the magnetic bead solution to the plasma sample, which is a first ratio, and performing uniform mixing;
performing centrifugation, placing the plasma sample mixed with the magnetic bead solution on a magnetic frame, and standing for 1 min-10 min;
removing a supernatant;
adding a first washing solution, standing, removing a supernatant after the supernatant is clarified, and repeating the above operations for multiple times; and
adding a second washing solution, performing uniform mixing, performing centrifugation, placing the centrifuged material on a magnetic frame for 1 min-10 min, removing a supernatant after the supernatant is clarified, and repeating the above operations for multiple times.

Step S500, separating the magnetic beads from the nucleic acid.

In particular, separating the magnetic beads from the nucleic acid may include:
adding an eluent, performing uniform mixing, and standing for 5 min-20 min; and
performing centrifugation, and placing the centrifuged material on a magnetic frame for 1 min-10 min to obtain a supernatant.

In order to more clearly understand the nucleic acid extraction method provided by the embodiments of the disclosure, further illustration is made as follows.

### Embodiment 1

A buffer system configuration is provided as follows:
(1) a digestion solution: 3.5 M GITC (guanidinium isothiocyanate), 20% Tween 20, 50 mM Tris (pH 8.0), and 25 mM EDTA;
(2) Proteinase K (20 mg/ml): 20 mg/mL proteinase K, 50 mM Tris (pH 8.0), 3 mM CaCl₂), and 50% by volume glycerol;
(3) a binding solution: 3.5 M GITC (guanidinium isothiocyanate), 45% isopropanol, 2.5% Tween 20, 10 mM Tris (pH 8.0), and 1 mM EDTA;
(4) First washing solution: 3 M GITC (guanidinium isothiocyanate), 30% isopropanol, 5% Tween 20, 40 mM Bis-Tris (pH 6.0), and 2 mM EDTA;
(5) Second washing solution: 50 mM Tris pH 8.0, 0.5 mM EDTA, and 80% by volume EtOH (ethanol); and
(6) Eluent: 10 mM Tris-HCl, and 0.1 mM EDTA, i.e., a TE solution.

### Embodiment 2

A buffer system configuration is provided as follows:
(1) a digestion solution: 1 M GITC (guanidinium isothiocyanate), 10% Tween 20, 30 mM

Tris (pH 8.0), and 10 mM EDTA;
(2) Proteinase K (10 mg/ml): 10 mg/ml proteinase K, 30 mM Tris (pH 8.0), 1 mM CaCl₂), and 15% by volume glycerol;
(3) a binding solution: 2 M GITC (guanidinium isothiocyanate), 20% isopropanol, 1% Tween 20, 10 mM Tris (pH 8.0), and 1 mM EDTA;
(4) First washing solution: 1 M GITC (guanidinium isothiocyanate), 10% isopropanol, 1%

Tween 20, 10 mM Bis-Tris (pH 6.0), and 1 mM EDTA;
(5) Second washing solution: 50 mM Tris (pH 8.0), 0.5 mM EDTA, and 80% by volume EtOH (ethanol); and
(6) Eluent: 10 mM Tris-HCl, and 0.1 mM EDTA, i.e., a TE solution.

### Embodiment 3

A buffer system configuration is provided as follows:
(1) a digestion solution: 6 M GITC (guanidinium isothiocyanate), 30% Tween 20, 100 mM Tris (pH 8.0), and 100 mM EDTA;
(2) Proteinase K (30 mg/mL): 30 mg/ml proteinase K, 100 mM Tris (pH 8.0), 5 mM CaCl₂), and 55% by volume glycerol;
(3) a binding solution: 5M GITC (guanidinium isothiocyanate), 50% isopropanol, 5% Tween 20, 100 mM Tris (pH 8.0), and 5 mM EDTA;
(4) First washing solution: 5 M GITC (guanidinium isothiocyanate), 50% isopropanol, 8% Tween 20, 100 mM Bis-Tris (pH 6.0), and 8 mM EDTA;
(5) Second washing solution: 50 mM Tris (pH 8.0), 0.5 mM EDTA, and 80% by volume EtOH (ethanol); and
(6) Eluent: 10 mM Tris-HCl, and 0.1 mM EDTA, i.e., a TE solution.

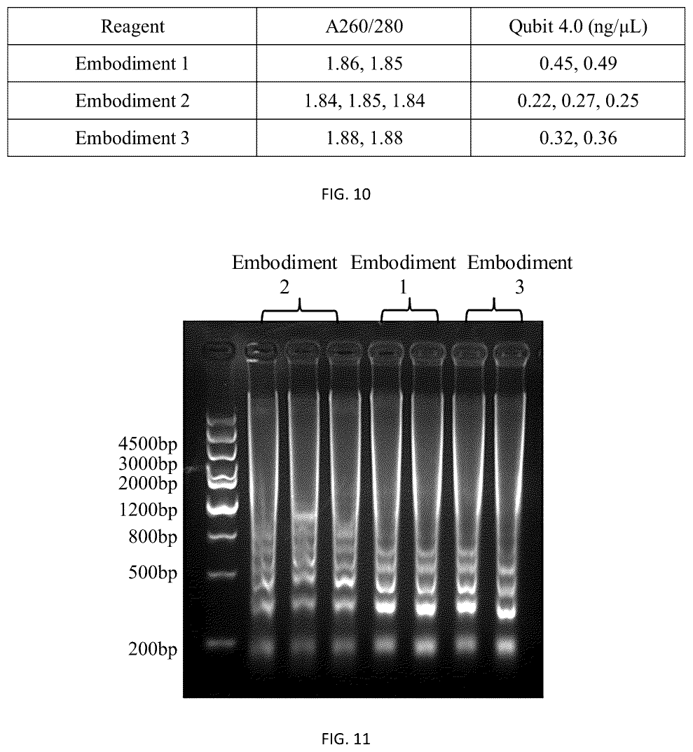

Extraction is performed by using the nucleic acid extraction compositions in Embodiments 1, 2, and 3 according to the following extraction operation process, a volume of the eluent is 40 μl, and the extracted DNA samples were subjected to Qubit 4.0 test, agarose gel electrophoresis test, and nanodrop test, respectively, and the results are shown in FIG. 10 and FIG. 11.

A specific extraction operation process was as follows.

### I. Preparation of Cell-Free Plasma:

1. a blood sample was centrifuged at 4° C. at 2000×g for 10 min;
2. the centrifuged plasma was transferred to a new centrifuge tube, being careful not to take a lower adherent yellow layer when a liquid was taken;
3. a plasma sample was centrifuged at 4° C. at 16000×g for 10 min or 6000×g for 30 min; and
4. a supernatant was transferred to a new tube for lysis with proteinase K in a next step.

### II. Proteinase K Digestion:

1. the plasma solution obtained in (I) was transferred to a 15 mL centrifuge tube, 0.02× proteinase K reaction solution (with a volume of 0.02 times the volume of the plasma sample) was added, and uniform mixing was performed by pipetting up and down;
2. 0.65× digestion solution (with a volume of 0.65 times the volume of the plasma sample) was added to the solution obtained in step 1, uniform mixing was performed, and the obtained mixture was incubated at 56° C. for 30 min to separate proteins from nucleic acids in the plasma sample; and
3. after incubation, the sample was placed on ice to be cooled for 5 min, or wait to be cooled to room temperature; and magnetic beads were added after cooling, which can avoid the problem that the magnetic beads is easily caked under the action of a magnetic frame during heating, which is not conducive to the re-dispersion of the magnetic beads into a washing solution and inconvenient for experimenters to carry out subsequent operations.
   III. Binding Magnetic Beads to cfDNA
1. a binding solution of which the volume was 1.5× the volume of an initial plasma sample solution (1.5 times the volume of the plasma sample) was added into the sample solution in (II), and uniform mixing was performed by vortex shaking to expose an organic group in the nucleic acid by isopropanol in the binding solution;
2. a magnetic bead solution was added into the above liquid to get a volume percentage ratio of magnetic beads to the plasma sample solution being 1:100, uniform mixing was rapidly performed on vortex, the obtained mixture was shaken at 1000 rpm for 10 min on a shaker to bind silicon hydroxyl of the magnetic beads to the organic group of the nucleic acid, so that the magnetic beads were linked to the nucleic acid;
3. centrifugation for a short time was performed, and a sample solution was placed on a magnetic frame to stand for 5 min;
4. a supernatant was removed, taking care not to disturb the magnetic beads;
5. a first washing solution, i.e., 1 mL of the first washing solution was added, and gentle pipetting up and down was performed by a pipette, so that all the magnetic beads are detached from a wall of the tube;
6. the magnetic bead dispersion solution in step 5 was transferred to a 1.5 mL centrifuge tube to stand for 2 min until a supernatant is clear;
7. the supernatant was removed without disturbing the magnetic beads;
8. operations in steps 5-7 were repeated once;
9. 1 mL of a second washing solution was added, uniform mixing was performed by pipetting up and down, and the obtained mixture was centrifuged;
10. the centrifuged material was placed on a magnetic frame to stand for 2 min until a solution is clear;
11. a supernatant was removed without disturbing the magnetic beads, and the steps 9-10 were repeated once; and
12. standing at room temperature for 5 min until there was no obvious ethanol solution on the surfaces of the magnetic beads for a reaction in a next step.
   IV. Elution of cfDNA:
1. an eluent with the same volume as the original magnetic bead solution was added into the centrifuge tube in (III), uniform mixing was performed by vortex shaking, and the obtained mixture was allowed to stand for 10 min (shaking up and down for 10 times every other two minutes);
2. centrifugation was performed on the 1.5 mL centrifuge tube in step 1, and the centrifuge tube was placed on a magnetic frame for 2 min after centrifugation; and
3. a supernatant was transferred to a new 1.5 mL centrifuge tube for next test.

As shown in FIG. 10, A260/280 represents the purity of cfDNA extracted and Qubit 4.0 represents the concentration of cfDNA extracted, data of A260/280 illustrates that the nucleic acid extraction composition provided by the embodiments of the disclosure is feasible, with ratios close to 1.8; data of Qubit 4.0 illustrates that the nucleic acid extraction composition in Embodiment 1 has a high extraction rate, and reagent component proportioning in Embodiment 1 was better. In FIG. 11, positions with higher brightness correspond to more nucleic acid fragments. As can be seen from FIG. 11, compared with Embodiment 2 and Embodiment 3, for the ratio of the nucleic acid extraction composition provided by Embodiment 1, the brightness was higher in the range of corresponding nucleic acid fragments, the nucleic acid extraction rate was higher, and the extraction effect of small nucleic acid fragments was also better.

In the embodiments of the disclosure, the buffer system 2 includes the digestion solution 21 and the binding solution 22 which are separated from each other, during extraction of a nucleic acid in a blood sample by using the nucleic acid extraction composition, cells in a plasma sample may first be lysed via the chaotropic salt in the digestion solution 21 for subsequent extraction of a nucleic acid in the cells, the nucleic acid is separated by the protease solution 23, then the binding solution 22 is added, the organic group in the nucleic acid can be exposed by the resolvent in the binding solution 22, the magnetic bead solution 1 is then added, the first group 12 linked to the magnetic beads 11 in the magnetic bead solution 1 can bind to the organic group, thereby linking the nucleic acid to the magnetic beads 11, based on the adsorption of the magnetic beads 11, other substances may be removed from the blood sample by washing and finally a relatively pure nucleic acid is obtained, thus realizing the separation of the nucleic acid from the blood sample. In the embodiments of the disclosure, the digestion solution 21 is separated from the binding solution 22, and the digestion solution 21 and the binding solution 22 are added separately for a reaction, so that a heating process during which the digestion solution 21 is added is separated from a process of binding the magnetic beads 11 to the nucleic acid by adding the binding solution 22, and the problem that the magnetic beads 11 attract each other during the heating process, impurities are not easily removed by washing in a later stage, and then the obtained nucleic acid has a low purity is avoided. Moreover, the binding solution 22 contains the resolvent, and the resolvent can expose the organic group in the nucleic acid, so that the magnetic beads 11 are linked to the nucleic acid by binding the organic group to the first group, and this binding allows to extract large nucleic acid fragments and to extract small nucleic acid fragments, ameliorating the disadvantage that only large nucleic acid fragments can be extracted in the related art. In addition, the digestion solution and the binding solution have different components and are separated, facilitating adjustment of a ratio of addition of the digestion solution and the binding solution according to blood samples of different volumes, improving the problem of poor operability on the addition volume of extraction solution for nucleic acid extraction under different blood sample volumes.

Although the preferred embodiments of the disclosure have been described, those skilled in the art may make additional changes and modifications to these embodiments once the basic inventive concept has been learned. Therefore, the appended claims are intended to be explained as including the preferred embodiments and all changes and modifications falling within the scope of the disclosure.

Obviously, those skilled in the art can make various changes and modifications to the embodiments of the disclosure without departing from the spirit and scope of the embodiments of the disclosure. Thus, if these changes and modifications of the embodiments of the disclosure fall within the scope of the claims of the disclosure and their equivalents, the disclosure is also intended to include these changes and modifications.

1. A nucleic acid extraction composition, comprising:
a magnetic bead solution, comprising magnetic beads and a first group linked to the magnetic beads; and
a buffer system, comprising a digestion solution, a protease solution and a binding solution;
wherein the digestion solution is separated from the binding solution;
the digestion solution comprises a chaotropic salt; and
the binding solution comprises a resolvent for exposing an organic group in a nucleic acid to allow the organic group to bind to the first group during nucleic acid extraction.

2. The nucleic acid extraction composition according to claim 1, wherein the resolvent comprises isopropanol, and the first group comprises silicon hydroxyl.

3. The nucleic acid extraction composition according to claim 1, wherein the digestion solution further comprises a chelating agent, a surfactant, and a buffer salt.

4. The nucleic acid extraction composition according to claim 3, wherein in the digestion solution, a concentration of the chaotropic salt is in a range of 1 M to 6 M, a concentration of the chelating agent is in a range of 10 mM to 100 mM, a volume percentage of the surfactant is in a range of 10% to 30%, a concentration of the buffer salt is in a range of 30 mM to 100 mM, and a pH of the buffer salt is in a range of 7.5 to 8.5.

5. The nucleic acid extraction composition according to claim 3, wherein the binding solution further comprises a chaotropic salt, a chelating agent, a surfactant, and a buffer salt.

6. The nucleic acid extraction composition according to claim 5, wherein in the binding solution, a concentration of the chaotropic salt is in a range of 2 M to 5 M, a volume percentage of the resolvent is in a range of 20% to 50%, and a concentration of the chelating agent is in a range of 1 mM to 5 mM; a volume percentage of the surfactant is in a range of 1% to 5%; and a concentration of the buffer salt is in a range of 10 mM to 100 mM, and a pH of the buffer salt is in a range of 7.5 to 8.5.

7. The nucleic acid extraction composition according to claim 1, wherein the protease solution comprises proteinase K, a buffer salt, a soluble salt, and glycerol.

8. The nucleic acid extraction composition according to claim 7, wherein in the protease solution, a concentration of the proteinase K is in a range of 10 mg/ml to 30 mg/ml; a concentration of the buffer salt is in a range of 30 mM to 100 mM and a pH of the buffer salt is in a range of 7.5 to 8.5, and a concentration of the soluble salt is in a range of 1 mM to 5 mM; and a volume percentage of the glycerol is in a range of 15% to 55%.

9. The nucleic acid extraction composition claim 1, wherein the buffer system further comprises a first washing solution and a second washing solution; wherein
the first washing solution comprises: a chaotropic salt, the resolvent, a surfactant, a buffer salt, and a chelating agent; and
the second washing solution comprises a buffer salt, a chelating agent, and an alcohol.

10. The nucleic acid extraction composition according to claim 9, wherein in the first washing solution, a concentration of the chaotropic salt is in a range of 1 M to 5 M, a volume percentage of the resolvent is in a range of 10% to 50%, and a concentration of the chelating agent is in a range of 1 mM to 8 mM; a volume percentage of the surfactant is in a range of 1% to 8%; and a concentration of the buffer salt is in a range of 10 mM to 100 mM, and a pH of the buffer salt is in a range of 5.5 to 6.5; and/or
in the second washing solution, a concentration of the buffer salt is in a range of 45 mM to 55 mM and a pH of the buffer salt is in a range of 7.5 to 8.5, a concentration of the chelating agent is in a range of 0.4 mM to 0.6 mM, and a volume percentage of the alcohol is in a range of 75% to 85%.

11. (canceled)

12. The nucleic acid extraction composition according to claim 1, wherein the buffer system further comprises an eluent;
wherein the eluent comprises: enzyme-free water or a TE buffer.

13. The nucleic acid extraction composition according to claim 1, wherein the chaotropic salt comprises guanidinium isothiocyanate, guanidinium thiocyanate, guanidinium hydrochloride, urea, sodium iodide, potassium iodide, potassium chloride, sodium perchlorate or trichloroacetate;
the chelating agent comprises: ethylene diamine tetraacetic acid, citrate, ethylene glycol bis (2-aminoethyl ether) tetraacetic acid, and cyclohexanediaminetetraacetic acid;
the surfactant comprises Tween 20, Tween 21, Tween 40, Tween 60, Tween 80, Tween 85, polyethylene glycol octylphenyl ether, ethylphenylpolyethylene glycol, polyoxyethylene lauryl ether, or sorbitan monolaurate; and
the buffer salt comprises: tris (hydroxymethyl) aminomethane or methylmethane.

14. The nucleic acid extraction composition according to claim 13, wherein the chaotropic salt comprises guanidine isothiocyanate;
the chelating agent comprises: ethylene diamine tetraacetic acid;
the surfactant comprises: Tween 20; and
the buffer salt comprises: tris (hydroxymethyl) aminomethane or methylmethane.

15. The nucleic acid extraction composition according to claim 1, wherein the magnetic beads have a size ranging from 200 nm to 5 μm.

16. A nucleic acid extraction device, comprising the nucleic acid extraction composition according to claim 1.

17. A nucleic acid extraction method, comprising:
obtaining a plasma sample according to a blood sample to be tested;
adding a protease solution and a digestion solution into the plasma sample, carrying out a reaction under heating for a first period of time, and performing cooling to separate a nucleic acid from the plasma sample;
adding a binding solution to the plasma sample from which the nucleic acid is separated to expose an organic group in the nucleic acid by a resolvent in the binding solution;
adding a magnetic bead solution into the plasma sample exposing the organic group to link the nucleic acid to the magnetic beads by binding a first group linked to magnetic beads in the magnetic bead solution to the organic group, so as to separate the magnetic beads linked to the nucleic acid from the plasma sample; and
separating the magnetic beads from the nucleic acid.

18. The nucleic acid extraction method according to claim 17, wherein the adding the protease solution and the digestion solution into the plasma sample, carrying out the reaction under heating for a first period of time, and performing cooling comprises:
adding the protease solution into the plasma sample, and performing uniform mixing;
adding the digestion solution into the plasma sample in which the protease solution is added, performing uniform mixing, and performing incubation at a temperature of 40-60° C. for 20 min-40 min; and
placing the plasma sample after the incubation on ice to be cooled for 1 min-10 min, or wait to be cooled to room temperature.

19. The nucleic acid extraction method according to claim 17, wherein the adding the magnetic bead solution into the plasma sample exposing the organic group to link the nucleic acid to the magnetic beads by binding the first group linked to the magnetic beads in the magnetic bead solution to the organic group, so as to separate the magnetic beads linked to the nucleic acid from the plasma sample comprises:
adding the magnetic bead solution at a volume percentage ratio of the magnetic bead solution to the plasma sample, which is a first ratio, and performing uniform mixing;
performing centrifugation, placing the plasma sample mixed with the magnetic bead solution on a magnetic frame, and standing for 1 min-10 min;
removing a supernatant;
adding a first washing solution, standing, removing a supernatant after the supernatant is clarified, and repeating above operations for multiple times; and
adding a second washing solution, performing uniform mixing, performing centrifugation, placing the centrifuged solution on a magnetic frame for 1-10 min, removing a supernatant after the supernatant is clarified, and repeating above operations for multiple times.

20. The nucleic acid extraction method according to claim 19, wherein the separating the magnetic beads from the nucleic acid comprises:
adding an eluent, performing uniform mixing, and standing for 5 min-20 min; and
performing centrifugation, and placing the centrifuged solution on a magnetic frame for 1 min-10 min to obtain a supernatant.

21. The nucleic acid extraction method according to claim 19, wherein the obtaining the plasma sample according to the blood sample to be tested comprises:
centrifuging the blood sample at a temperature of 1° C.-10° C. for 5 min-20 min;
obtaining a supernatant after the centrifuging to form an initial plasma sample;
centrifuging the initial plasma sample at a temperature of 1° C.-10° C. for 5 min-50 min; and
obtaining a supernatant to form the plasma sample.
